(19) United States
(12) Patent Application Publication
(10) Pub. No.: US 2022/0198576 A1
(43) Pub. Date: Jun. 23, 2022

(54) SYSTEMS AND METHOD TO TREAT COMPLEX REVENUE CYCLE MANAGEMENT TRANSACTIONS WITH MACHINE LEARNING IN A STANDARDS-BASED ELECTRONIC DATA INTERCHANGE

(71) Applicant: Mimeograph Holdings, LLC, Boca Raton, FL (US)

(72) Inventor: Daniel William Shields, Boca Raton, FL (US)

(73) Assignee: Mimeograph Holdings, LLC, Boca Raton, FL (US)

(21) Appl. No.: 17/131,269

(22) Filed: Dec. 22, 2020

(51) Int. Cl. G06Q 40/00; G06N 20/00; G06Q 40/08; G06F 16/14

(52) U.S. Cl. CPC G06Q 40/12; G06N 20/00; G16H 10/60; G06F 16/156; G06Q 40/08

(57) ABSTRACT

A computer system comprising input from a plurality of data sources, performing analysis, and configured to select and apply multiple machine learning algorithms in a conduit of components to predict cost reimbursement probability and predict figure, billable line items per terms, and recommend transaction code input, order and formats. When invoked by request from an integrated client, a recommendation is retrieved by processing a query service from the system through application program interfaces and presented to a user. User acceptance and quantifiable outcomes of each transaction are used as supervisory and reinforcement functions to iterate and improve prediction, and outcomes; through subsequent applied models to monitor evolutions of system input considerations. A digital application layer provides a visual interface for the configuration of the machine signals which modulate system performance, and exhibits reporting on measurements tied to specific decisions to deliver a recommendation.

### TECHNICAL FIELD

The present invention relates to systems and methods for capture and analysis of individual patient healthcare records for processing with the intent of helping recommend service and hardware billable cost inputs for electronic transmission to third party systems, and subsequent payment.

### BACKGROUND

In a medical procedure transaction, precise aspects of the treatment plan are decided by one or many qualified medical professionals. A variety of treatment options may exist, and the materials and services needed to carry out these procedures adhere to evolving coding standards related to: diagnostic conclusions, required expertise for executing or carrying out the treatment, standard and special materials, tools and implements, as well as time and space within a facility to operate. As complexity of an episode of care increases, so too does the likelihood of billing and claim submission errors and the subsequent reduction in or whole denial of payment for services by a patient insurance carrier. Thus, exposing the patient, medical professional, facility, materials supplier and insurance carrier to additional and compounding risk.

The risk includes large volumes of non-payment and substantial delay in payment on remaining balances transferred to a patient, or their families, for their treatment. As the risk in the shared economy grows, so will the costs and complexity to absorb the risk.

The problem is most immediately affecting the buyer-seller relationship in the medical field due to the masked nature of the cost alternatives for patient's which live behind the assessment process and decisions contributed by medical professionals. Therefore, it is desirable to provide collaboratively sourced information about results from volumes of diverse treatment plans to supply high confidence recommendations about the decided treatment plan, and the subsequent economic impact to all affected parties as to improve outcomes, reduce risk, and expose affordable options to communicate to the patient (or financially responsible party) whenever possible.

After research and development; including substantial searches for prior art or existing publications, there does not appear to be description and detail sufficient to challenge the uniqueness of this invention. While unique in its application; the subject invention of this disclosure claims uniqueness by arrangement and improvement upon existing prior art and cites significant research to determine uniqueness. There are existing novel technologies, or concepts reduced to method, which describe machine learning applied in a variety of contexts. Some include the ability to leverage medical input data to present data to insurance companies for ‘pre-authorization’.

This is sufficiently different in concept and application. Additionally, technologies exist which seek to classify and evaluate the value of services provided to a patient for reporting purposes for analysis and making strategic recommendations about improving outcomes. All researched existing and published art stops short of implementing a technical closed loop solution to automate ad hoc recommendations about affordable treatment plans with the help of any, less diverse applications of machine learning technologies.

No prior art or publication surfaced during exhaustive efforts to locate existing systems and methods yielded sufficient classification and arrangement of technology to merit reconsideration of filing.

With the current popularity and research dedicated to applied machine learning functions of business, there is significant art within the marketplace and in pending and granted patent filings related to the subject.

The preventative nature of the volume of data needed to operationalize machine learning models is exhibited by the fact most attempts fail to demonstrate interoperability to source records from a plurality of facilities across a topology to access sufficient diversity of input. Thus, the subject invention is differentiated by the ability to consume standards-based interchange of data about the claims and payment details, as well as access infrastructure to source inputs needed from FHIR and EDI. The subject invention can access a sufficient volume across a subscriber topology with semantic interoperability activated for the means to process predictable fields from diverse contributing external sources, across standard interchange formats, to gain improvement while scaling growth of considered records and inputs.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

In one embodiment of the present invention, a system comprises a data digest component, an aggregation component, an extract-transform-load component, a data treatment component, a database component, a computation network component, a graph modulator component, a machine-learning graph component, and a server-side application program interface component.

The data digest component is configured to move data from a plurality of server systems within a secured service unit into a receiving server folder structure, across an encrypted communication data protocol conduit for interchange. The aggregation component is configured to temporarily store data from the digest in a general file system. The organization component is configured to rename and store files according to their origin and contents for use and to individually parse each file and store the contents of the file in a database component. The routing component is configured to process and analyze the data records in the database component to build stored procedures for access by any of several node tenants, in a network of virtual server machines, for intricate processing and supervised-feedback iterations. Each of the individual node tenants components are configured to execute a specific formula for comparison and evaluation on the efficacy of machine-learning treatment on the records stored within the database component. Each node constitutes a configured virtual machine configured to generate a representative relational data graph storing keyed and structured lookup by comparative input matrix. A server-based software program component is configured to use and select from the nodes the optimal mix and order for inputs of processing for the requests fielded by the core central tenant software application from each installation of an externally integrated digital application client.

In a second embodiment, a system comprises an integrated digital input form component, a client-based application program interface software component, a digital server authentication component, a transaction query component, an API request constructor component, a visualization and interactive selection user-facing component, and a supervisory input selection component. The integrated digital input form component is configured to collect real input data about the details of a complex transaction.

The client-based application program is configured to construct a limited set of server-requests related to the details of the complex transaction, to the application server endpoint described in the first system embodiment. The digital server authentication component is configured to handle presenting precise credentials to the externally controlled client technology in which it is integrated as well as configured to handle authentication with proprietary software systems. The visualization and interactive selection component is configured to provide usable functionality to review the results of the calculations returned by System One in a format for interpretation and analysis by the user, and subsequent selection of the optimal transaction for all parties participating in the exchange. The supervisory input selection component is configured to capture the selection and qualities about the input criteria from System Two and generate a subsequent request to System One to generate record of the factors influencing the selection and presenting new information to specific tenants in System One.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates the end to end binary system integration of the current invention on an exemplary EHR-S application installed in a hospital, urgent care center, skilled nursing facility or other healthcare services provider, according to some embodiments;

FIG. 2 illustrates a block diagram of the secure file transfer component and data security treatment system, according to some embodiments; and

FIG. 3 illustrates a block diagram of the VPS/Cloud application interfacing, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

While the specific scope of the embodiments is the use of these systems and methods to leverage standards to help billing and payment inefficiencies in the medical/healthcare industry, the application has sufficient foundation for broader protection. EDI (‘Electronic Data Interchange’) forms are used by CPG Retail (‘Consumer Packaged Goods’), logistics, transport, manufacturing and business services.

One skilled in the arts will readily understand that the embodiments may be applied to a variety of industries which require multiple parties during the task or project planning, execution, billing, and payment processes. Due to the integrated nature of the invention and by reference to the EDI Standard, a likely application of the same technology with no core modification would functionally deliver similar performance based on regulated and standard EDI form consumption; and thus, should be considered within the scope and field of the protections sought.

Some applicable industries wherein the embodiments provided herein may be utilized include the financial services, healthcare, logistics, automotive, and travel. The embodiments allow businesses within various industries to communicate and collaborate throughout their various business processes.

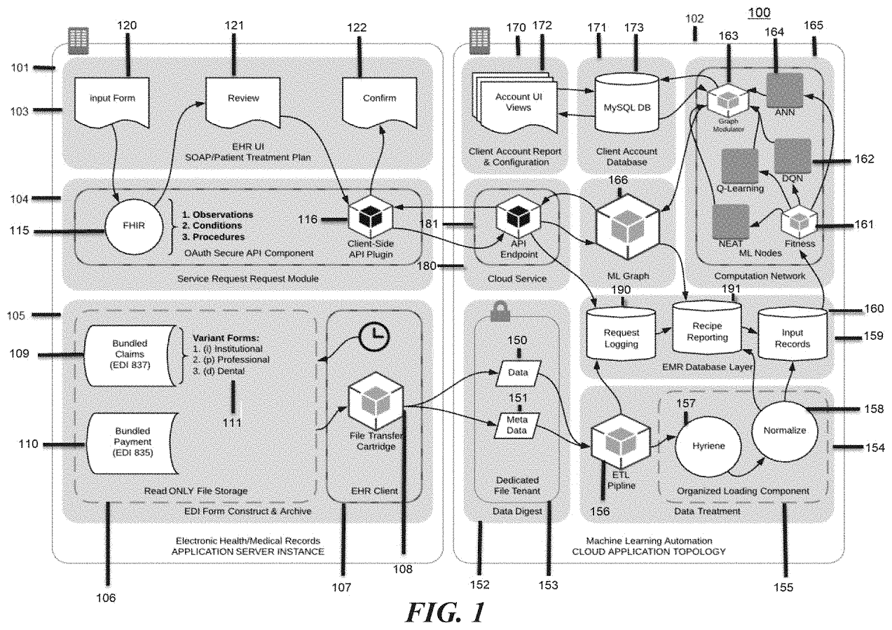

In reference to FIG. 1, individual healthcare professional service providers (doctors, nurse, skilled medical staff or attendants), healthcare institutions and consolidated healthcare networks (or groups) have established a significant infrastructure for the use and interchange of patient related data for real-time and near-real time access to information. These are distributed software applications which are centrally governed within the hospital or practice for the purpose of evaluating and documenting the state and billable services or a treatment plan. Data is stored in a dedicated or semi-shared network within the hospital with security protocol and restrictive access. This access and data are related to their customer (“patient”). The nodes on this network are individually implemented, installed or integrated EMR (“Electronic Medical Records”) or EHR (“Electronic Health Record”) systems 101. These systems adhere to specific standards for the purpose of interoperability. This to say: there is expected native exchange of data between individual platform installations regardless of the underlying software or operating system. For the purpose of this invention, we'll refer to the variable capacity for installation in multiple systems and design patterns, as one of being ‘platform agnostic’.

At a given moment or state within a patient episode of care, once established through medical protocol, an input about the diagnostic state of the patient's care and a state of treatment is presented within billing and associated code formats. Codes exist in multiple formats to carry out tasks of itemizing the condition and diagnosis of the patient and to ensure that materials associated with the procedures carried out to treat the patient are detailed according to a standard.

These may include historical references to previous treatment dates, varied and detailed taxonomy associated with the specific cause and condition being treated, or a number of approved methods, prescription, and/or hardware which contribute to the

application of medical sciences to alleviate pain and encourage or promote positive restoration of health.

For the purpose of this invention, the codes which are used for billing are stored within an Bundled Claims (EDI 837) 109 File format at a designated secure position within a restricted-access file structure residing in the APPLICATION SERVER INSTANCE [101]. As is illustrated by the listed formats comment 111, there are forms which vary slightly (though not enough for the purpose of this invention) and provide classification for claims originating from Institutions (Inclusive of but not exclusive to hospitals, urgent care centers, skilled nursing facilities) 111,1, Professional Services Providers 111,2 (Surgeons, Residents, Specialists, Attending Physicians, and Dentists 111,3. These comprise the entire scope of INPUT consideration for the backend data interface across the EpisodlQ EHR Client 107.

The event triggered or time based transfer of the files is carried out by a piece of software called the File Transfer Cartridge 108 which can detect new files and transmit them in real-time upon creation, or in timed routines to a Dedicated File Tenant 153 within the Data Digest component 152. This component is configured to validate the file contents and appends meta data about the origin and qualities about the contents of the file for use in organization by a Data Treatment Component 154. The Dedicated File Tenant 153 therefore also acts as a security perimeter for the data and as an extended component to act within a firewall until the records transformation takes place and transmission to the ETL Pipeline has completed 156.

The Data 150 and Meta Data 151 contained in the Bundled claims 109 and Bundled Payment 110 files are exposed to a processing script for the purpose of Extracting, Transformation and Load (ETL) 156 into subsequent stages. At this point, the data affiliated with any personally identifiable information (PII) or HIPAA (Healthcare Insurance Portability and Accountability Act) designated field is obfuscated by secure and restrictive field level encryption for any storage or read thereafter. This can be done with the aid of a ‘Blockchain’ for retrieval and validation, but is

not claimed essential for this invention. Unused or unnecessary storage of personal details of treatment are thereby securely encrypted end to end, and inaccessible to any user on the CLOUD APPLICATION TOPOLOGY 102.

During transfer of the data fields across the ETL Pipeline 156, a log record 190 is captured about the Data 150 and Meta Data 151 which are stored for the purpose of gathering statistics to improve function, to research and account for data routine efficiency, and to provide a record of each file which is considered by the system. This is done for the benefit of forensic purposes as well, should a need to correct, purge or report on the specific details exists with a record of provenance. This feature is also developed for the anticipation of future design and compliance requirements related to security and privacy of systems where users are likely to exchange personal information related to health, treatment, and commercial transaction details. Data for logs are stored within the EpisodlQ Machine Record Database Layer (EMR Database Layer) 160 in a separate database considering event logs 190.

Once the ETL process has performed the initial task of creating an input record set for the contents of the EDI files, the intermediate state is semi-structured and exposed to a Hygiene 157 and Normalize 158 Process in the Organized Loading Component 155 of the Data Treatment facility 154. Here data is run through routines of processing intended to increase the quality and uniformity of data which will be transferred into the EpisodlQ Machine Record (EMR Database Layer) 159. Data is read and cleaned of character mutations and prescribed field and format treatment for data types which lend to efficient storage and retrieval of records by the map reduced database system. Upon completion of these two functions, data is inserted into a Reporting Recipe Database 191 within the EMR Database Layer 160 and stored in full record format within the Input Records Database.

The Input Records Database 160 is a structured large volume storage facility which can be queried directly by the individual Machine Learning Nodes 164 within the Computation Network Component 165.

As a traffic agent to the data which is being collected, emulation routines are run periodically through a software element which conducts statistical analysis related to application of specific Machine Learning Algorithms 162 which can be applied for Fitness 161 for the type of optimization being conducted. Based on feedback from the systems at large, and the outcomes presented by application of the specific machine learning methods represented by these nodes, data is shared across the topology of nodes in the Computation Network 165. Continuity of the data outcomes of the application node calculations are then directed by editorial control of frequency and weights of considered variables by a Graph Modulator 163.

The Graph Modulator enables editorial control over the ML Graph, which is exercised by user inputs on technical view states in an Account User Interface 172 which allows visibility and control over the machine learning application by way of Client Account Report & Configuration 170 component. These are handled again by a smaller, distributed application instance 170 per installation. This application is secured by authentication of users with Access Control Roles contained within a local MySQL DB 173 instance governing models and access within the Client Account Database 171. Here a user can see the performance of the applied algorithms and the functions of the software, as well as use inputs to moderate adjustment thresholds and weight of application functionality by affecting variables considered by the Graph Modulator component.

Several designated Machine Learning Algorithms 162 are considered; each by an individual ML Node 164, within the Computation Network 165. Some instances incorporated by reference here include the use of Q-Learning 162, Artificial Neural Networks, Neuro-Evolutionary Augmenting Topologies, and Deep Query Networks. Each of these are at the same time experimental and subjective by their nature with certain implementation challenges. As such, each is implemented individually for computation on the same data for use in a comparative matrix that considers the benefits of application, reinforcement by outcomes and activities of the consumer, and the inputs of configurations for modulation by the objectives of the business entity subscribed to their implementation.

The Graph Modulator 163 prescribes the routines which generate the content of a large static file called a Machine Learning Graph (ML Graph) 166. This is a nested NOSQL data architecture which indexes recipes.

The ML Graph acts as an efficient lookup (or query) resource for input keys fielded by HTTP Request content payload fielded by the API Endpoint 181 in the EpisodlQ Cloud Service 180 component of the Cloud Application Topology 102. This static file will be generated when the critical mass and threshold for adjustment take place within the optimal mix algorithm within the Graph Modulator 163. In this invention, this happens based on predictions from inferential statistics related to the treatment of variables presented across the Dedicated File Tenant 153 within the Data Digest Component 152.

The ML Graph 166 is loaded by a software process which also keys logs and recipes through advanced indexing of each incarnation of the graph entity, and its contents stored in archive for use in subsequent analysis. These records are stored within the Recipe Reporting Database 191 within the EMR Database Layer 160.

When accessed by an authorized and authenticated request presented to the EpisodlQ API Endpoint, within the cloud service, the ML Graph 166 is queried with information presented in the HTTP(s) Request generated by an EpisodlQ Client-Side API Plugin 116 instance within the Service Request Module 104 component.

In order to generate a HTTP(s) Response with a recommendation from the CLOUD APPLICATION TOPOLOGY 102, a consumer within the EHR APPLICATION SERVER INSTANCE 101 has to generate an authorized HTTP(s) Request using the EpisodlQ Client-Side API Plugin 116. These are constructed by small procedure scripts which collect information about the current subject of a patient episode of care in specific view states of the EHR UI 103, exhibiting construction of a Patient Treatment Plan.

During a Patient Treatment Plan 103, a consumer of the EpisodlQ Cloud Service 180 interfaces with input forms in a multi-step transaction 103. At the outset, when a diagnosis is determined for the current patient subject, a user generates information tied to a MRN 120. As treatments are considered and ordered for the specific diagnosis of the patient and inputs are logged against the EHR 120. For each new and atomic input for the treatment plan designed by the qualified medical professional and their staff, a new HTTP Request is generated by the EpisodlQ Client-Side API Plugin (ECSAPI) 116 using input attached to the Health Level Seven (HL7) Fast Healthcare Interoperability Resource (FHIR) 115. The information used includes the patient Medical Record Number (MRN), and the ECSAPI collects data from FHIR in multiple methods.

Authentication for these applications requires a specific client key which is provided per installation of the ECSAPI. Authentication between the FHIR Resources and the client are governed by an accepted authentication method called OAuth 104. This allows for seamless and secure communication between the EHR installation of HL7's FHIR product, and the ECSAPI.

ECSAPI gathers input related to the patient episode of care in the form of reading three core methods. These include: Observations, Conditions, and Procedures respectively 115. The ESCAPI receives a HTTP(s) Response from the EpisodlQ Cloud Service 180 by way of presenting inputs on the HTTP(s) Request to the API Endpoint 181.

The JSON Payload of the response is then formatted by ‘Callback Functions’ within the ECSAPI and presented to the user for confirmation. These prescribe treatment and presentation of the HTTP(s) Response. In some EHR APPLICATION SERVER INSTANCES 101, this might include a module for visual representation against each recommendation for representation of potential outcomes with percentage

representation, potential critical cost thresholds for a patient plan, or color formatting to indicate potential problems with a given input.

Upon receipt of satisfactory HTTP(s) Response, a user is prompted in the final EHR view state considered in the scope of this invention. When a new treatment plan is adjusted with the financial considerations of the recommendations received and presented by the ECSAPI, acceptance presents a ‘Confirm View State’ 122 which subsequently affects the MRN Treatment Plan, and influences the contribution to an EDI 837 109 data file for submission to CMS, Insurance Carriers, and/or individually responsible payers. When payment advice is received associated with the individual claims as part of a bundle, electronic forms associated with payments are stored as Bundled Payment (EDI 835). These are placed in a representative state within the EDI Form Construct & Archive component of the APPLICATION SERVER INSTANCE.

Storage constitutes availability for pick up and consumption by the EpisodIQ EHR Client and thus closes the feedback loop necessary to automate the data and analysis needed to trigger recommendations generated by the Computation Network.

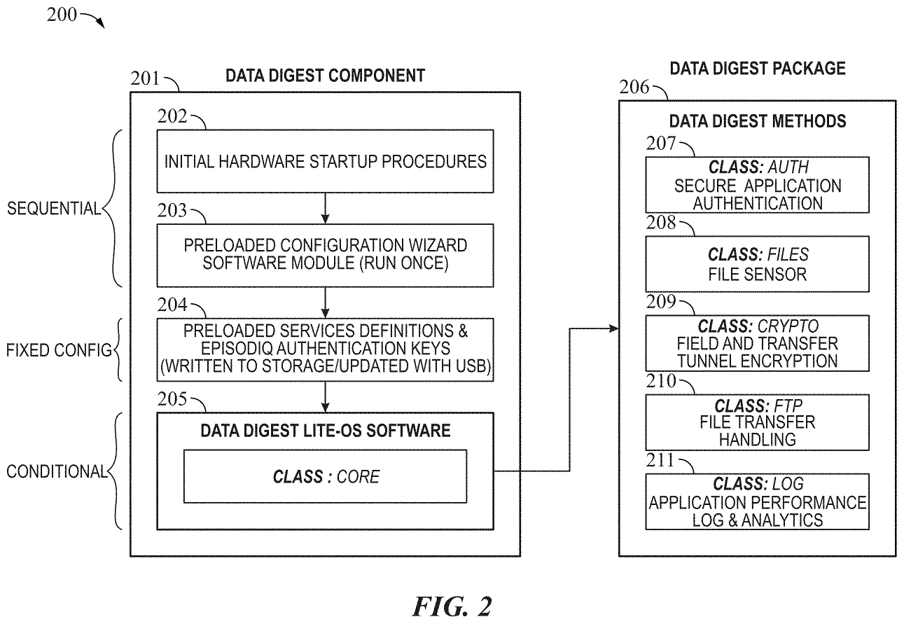

In reference to FIG. 2, a detail of the specific ‘Data Digest’ component which operates as an integrated software application preset to handle secure and encrypted transfer of the EDI 837 forms which are collected by the healthcare or medical EHR systems which organize and store medical records from individual treatment ‘Episodes’. This executes these commands according to defined protocol within the component which listen for new ‘complete’ files in the directory and handle a read-write operation to open the file, encrypt contents at the field level where PHI and PII is stored, and render the file across a secure and encrypted connection to a repository inside an authenticated server component. When a new file is read and the transfer is complete, a log is generated to the core application server which generates a new row in a log file and subsequently a new record in a governing application function feature to monitor system performance and volumes for reporting.

On a configured hardware device, the Data Digest component 201 exists as a minimally exposed operating system which was pre-programmed with integration keys for extension from a cloud-based application, as well as with the proper secure transfer credentials for the specific subscribed and active instance.

For the purpose of submission in the present invention, the micro-operating system has a preset set of instructions to handle compiling and preparing the operating system to receive commands. When initialization 202 is carried out on the operating system the application sequence is run and exhibits a series of views in a ‘software wizard’ 203. These define and validate the configurations of the local machine within the subscriber EHR systems network, and accepts input for further authentication with the encryption provider, and validates the required input/output connection to the servers.

Initial points of configuration include:
1. Authentication and saving credentials to the LAN or associated secure Wi-Fi network (where in either case a Network IP is assigned to the machine)
   a. For Secure Wireless Internet Broadcast Clients:
      (i) SSID Definition is located by local radio acquisition
      (ii) When SSID selection is made, user is prompted for password
      (iii) Input strings are written to the BIOS and OS Shell to save configuration
   b. For LAN Selection
      (i) A CAT5/CAT6 cable is connected to the Ethernet port on the hardware and mounted on the Local Area Nework
         1. Additional requirement for configuration here might include Firewall/Network whitelisting and validation sequences.
      (ii) The keys are written to the BIOS and OS Shell to save configuration for operational simplicity.
2. Locating mapped EDI form claims and payment files (837/835Forms) stored in a secure directory within the subscribing client EHR or health data storage as part of the subscriber network topology
   a. Simple user interface on prompting will allow the network drive and exact location to be defined by technician or by the subscriber through provisioned documentation.
   b. Due to variation on the locations and styles of storage for existing EHR standards, this step may vary.
3. Extending access to software packages and authenticating with the remote cloud server application.
   a. A logical test when the ‘Wizard’ 203 completes moves the operation to the next interface which is a branded view of the EpisodlQ system within the terminal 204
   b. With a prompt and confirmed insertion of the provisioned USB stick, data is loaded from the stick to the configuration script within the Data Digest component CORE methods class 205.
4. Executing whitelisting of the machine physical hardware address (MAC Address) and the range of IPs which are accepted by the Remote Cloud Server instance 207
5. Enabling and retrieving the certificate and credentials for an SFTP (Secure File Transfer Protocol) within the SSH2 exchange procedure 210
   a. This feature will require support and maintenance overt ime to update keys and certificates for access through the introduction of a new paired key certificate file or authentication certificate.
   b. For security purposes, these will be provided by installation via a password protected procedure running from software installed on a provisioned USB stick and directly updated within the system while personnel is present 204
6. Establishing 2-way encryption from the Data Digest component and the Remote Cloud Server Instance using field level encryption/decryption 209
   a. This particular feature uses industry current standard:AES-256; but we comment here to extend the scope to include such methods as fully leveraging a stable and secure Blockchain or Ledger technology such as ‘Elastos’ or ‘Ethereum’, both of which are respectively different brands and emerging approaches for this application. These are all handled within the AUTH 207 and CRYPTO 209 class methods.

When completed, the pre-compiled software application will operate a collection of packages which will handle the task of picking up a file for digest by the input side of the application. The packages have specific and limited tasks which operate out of a controlling file which has periodic and state-based conditions which build and manage an automated queue for processing in batch or in a singular file state.

In each singular instance where an 837 EDI OR 835 EDI, the FILES Class 208 of the Data Digest Package 206 operates to inventory existing files in the respective directories which were defined by the configuration steps in the ‘Wizard’ 203. During the initial setup phase, this procedure is also run to establish conventions and schema which will be used to aid in reliability and scalability to the file system in downstream consumption and learnings applications.

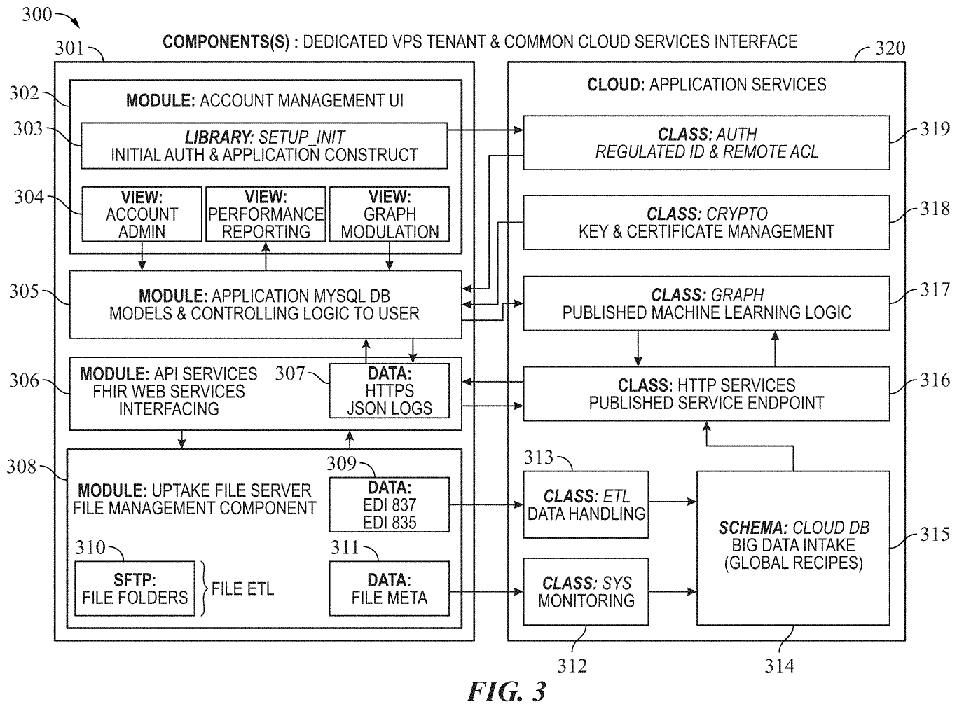

FIG. 3 describes the components of the present invention which constitute a complete single instance of the consumer facing Application Server 301 hosted and operating on a single and dedicated VPS Instance within a secure cloud network setting. This instance is natively supported by hosted Common Cloud Application Services 320 which securely interface with the data transformation moved through the Application Server. Each instance of the server acts as an account management interface for the uptake and sharing of files 310 as well as having refinement and fine tuning 304 of the operating algorithms within the Account Management UI 302. This provides the consumer with some level of control over the tasks related to augmentation of business duties with the help of these models and routines. Multiple components of the claims in this application are referenced here with close proximity to exhibit those which are a part of a single-instance stack in a dedicated consumer tenant; in contrast to those which are common and shared with ALL tenants for network regulation and logged authentication.

Upon initial load up of the hardware system in [FIG. 2], and authentication with the cloud application server, a build script is initiated within a new allocated and dedicated virtual private server instance. A library nested within the instance launch 303 provides instructions to the server to utilize information set into the configurations, and execute directives to build the directories and copy application files to render a server-side application with a full consumer user-interface.

The UI of the subject of this component within the current invention is meant to accomplish technical moderation and modulation of the attached machine learning network nodes to the optimal application of or preferences of the consumer. This is accomplished by providing three (but not limited to this figure) main views into the application 304. Upon accessing and completing a light form series to validate configurations and promote security settings to the group of users; the application authenticates 319 by communication with the cloud server module. The authentication and security keys for the account are stored with permissions limited into the instance data base located in the MySQL DB Module 305 of the dedicated tenant.

The dedicated server instance 301 also acts as the construct SFTP receiver from the Lite OS 205 which communicates to this instance natively within the FTP Class 210 in the available Data Digest Methods 206. This ensures native and secure transfer between the hardware—through a dedicated and consumer-controlled instance within their network firewall—and across to EpisodlQ's Cloud Server Application.

FIG. 3 also represents interface of the VPS with the EHR co-located FHIR Services 306 (Fast Healthcare Interoperability Resources) which allow the account to respond to requests made from within the EHR System Services via API to request recommendations about form inputs within the preparation phases of the Insurance Claim (EDI 837). An ad-hoc request is made for each input within the EHR which is made through a REST API 316 request using OAuth2. This request is handled by the API services which in turn gathers its response from the GRAPH 317 portion of the Cloud Application Services. The response is routed back to the EHR across the API Services Module 306 of the Account application, and rendered back to the EHR user in the form of a HTTPS JSON 307 payload signaling data which can be parsed by the client to help re-direct the billing inputs to optimally suitable transaction line items.

Changes can be made to how each healthcare consumer benefits from the machine learning models which power and inform the GRAPH 317 features. Through modulation and configuration of the performance, a consumer can manually interfere with weighting and other considerations where their business might benefit. Insights to inform modulation can be drawn from the ‘Performance Reporting View’ nested within the image 304.

Access control, privacy, compliance, anonymity of user data, and user-management are handled by features and functions which reside in the AUTH 319 and CRYPTO 318 software classes within the Common Cloud Services 320. These act as governing libraries for access and readability management which is keyed into the privacy and security keys configured in Data Digest Methods as part of the Data Digest Package 200 when the CORE Class 205 within the Data Digest Component 201 is initiated. Due to the nature of the crypto-logical bond between the hardware and the application, and out of an abundance of caution, the application is designed to provide tremendous access to detailed patient data ONLY to an intact tether on a security chain which binds these components to each other, while only sharing and promoting fully anonymous data to the Cloud Schema DB 314.

Data which is moved into the Cloud Application Services 320 common containers and objects is exposed to the ETL 313 which assures maximum match and hygiene while providing input FILE META 311 figures into the data schema for reporting back on functionality and uptake of the general system at large. In this construct, records associated with the entire network of individual subscribers and institutions can benefit from the consolidated and aggregated data collection across the Machine Learning Network Nodes 400 and render probabilities recipes to the GRAPH 317. This is also done for efficiency of the response back to the EHR operator. The input and governing settings and configurations for interpretation of that graph (having weights and preferences applied) is the loopback to performance reporting within the View of the Account Management UI 302.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system to centralize aggregated insurance claim details and payment advices, the system comprising:
a client-based integrated application program interface component to collect and organize a plurality of transaction line item data;
a services integration component to interconnect an active individual FHIR record to a plurality of selected individual goods and services records via MRN;
an integrated digital input form component which exchanges data with the client-based integrated application program interface from within a EHR treatment planning view;
a visualization component to interpret the feedback from the integrated digital input form and to display information for the end-user; and
a digital authorization component to interchange EDI transactions containing a plurality of information.

2. The system of claim 1, wherein the client-side API plugin authenticates against an EHR installation of the FHIR and collects the plurality of information.

3. The system of claim 2, wherein the plurality of information is collected by observations, conditions, and procedures.

4. The system of claim 1, wherein the visualization component displays information for the end-user by presenting one or more statistical figures.

5. The system of claim 1, wherein the visualization component displays information for the end-user by presenting one or more variable cost outcome scenarios.

6. The system of claim 1, further comprising a data digest component to identify, read, copy, rename, clean and transfer a copy of a standardized electronic data interchange file.

7. The system of claim 1, further comprising a data treatment component to organize data for each of a plurality of installed instances within a shared server environment.

8. The system of claim 1, further comprising an organization component configured to utilize one or more scripting methods to identify an origin and a plurality of attributes of the transaction data, and to classify the transaction data into a set of files and folder structures;

9. The system of claim 1, further comprising a server-based application program interface component which receives a plurality of requests and queries the relational graph through a plurality of analytical routines and subroutines determined by algorithms, and respond to the plurality of requests with a readable structure for an authorized client to interpret and display; and

10. The system of claim 1, further comprising a server application component to present a customer with a customer with a view of a plurality of performance algorithms in a report structure, the server application component to modify how a computation network interprets and applies the transaction data to the relational graph.

11. A system to centralize aggregated insurance claim details and payment advices, the system comprising:
a data digest component to identify, read, copy, rename, clean and transfer a copy of a standardized electronic data interchange file;
a data treatment component to organize data for each of a plurality of installed instances within a shared server environment;
a data file aggregation component to receive transfer of a transformed file format for the transaction data in an electronic file system on a secure centralized resource collection;
an organization component configured to utilize one or more scripting methods to identify an origin and a plurality of attributes of the transaction data, and to classify the transaction data into a set of files and folder structures;
a data extraction component to open, read, store, and load a plurality of selected information stored in the set of files into a database component, the database component to store and organize the transaction data from the set of files into an electronic format for query, analysis, reporting, and quality assurance;
a computation network component including a plurality of nodes;
a currency and exchange component to perform conversion of figures within a plurality of associated transaction records to express outcomes and computations in a localized market or a specific geography wherein an exchange occurs;
a relational graph format which organizes the transaction data into keyed recipes;
a server-based application program interface component which receives a plurality of requests and queries the relational graph through a plurality of analytical routines and subroutines determined by algorithms, and respond to the plurality of requests with a readable structure for an authorized client to interpret and display; and
a server application component to present a customer with a customer with a view of a plurality of performance algorithms in a report structure, the server application component to modify how a computation network interprets and applies the transaction data to the relational graph.

12. The system of claim 11, wherein the data treatment component is configured as a security perimeter for the data.

13. The system of claim 12, further comprising a dedicated file tenant configured as an extended component within a firewall until the records transformation takes place and transmission to the ETL pipeline has completed.

14. The system of claim 11, wherein the data is contained in the bundled claims.

15. The system of claim 14, wherein the bundled claims and a plurality of bundled payment files are exposed to a processing script for the purpose of extracting, transformation, and loading into subsequent stages.

16. The system of claim 15, wherein a log record is capture about the data to improve function efficiency, and provide a record of the data.

17. The system of claim 16, further comprising a reporting recipe database within the EMR database layer stored in full record format within an input records database.

18. The system of claim 11, wherein a graph modular prescribes routines to generate the content of a machine learning graph.

19. The system of claim 18, wherein the machine learning graph acts as a query resource for input keys fielded by an API endpoint.

20. A method to centralize aggregated insurance claim details and payment advices, the method comprising the steps of:
identifying, reading, copying, renaming, cleaning and transferring a copy of a standardized electronic data file, via a data digest component;
organizing, via a data treatment component, data for each of a plurality of installed instances within a shared server environment;
receiving, via a data file aggregation component, a transformed file format for the transaction data in an electronic file system on a secure centralized resource collection;
identifying, via an organization component, an origin and a plurality of attributes of the transaction data, and to classify the transaction data into a set of files and folder structures;
opening, reading, storing, and loading a plurality of selected information stored in the set of files into a database component, the database component to store and organize the transaction data from the set of files into an electronic format for query, analysis, reporting, and quality assurance;
converting, via a currency and exchange component, a plurality of associated transaction records to express outcomes and computations in a localized market or a specific geography wherein an exchange occurs;
organizing, via a relational graph format, the transaction data into a plurality of keyed recipes;
receiving, via a server-based application program interface component, a plurality of requests and queries the relational graph through a plurality of analytical routines and subroutines determined by algorithms, and respond to the plurality of requests with a readable structure for an authorized client to interpret and display; and
presenting, via a server application component, a customer with a customer with a view of a plurality of performance algorithms in a report structure, the server application component to modify how a computation network interprets and applies the transaction data to the relational graph.
